(19) United States
(12) Patent Application Publication
(10) Pub. No.: US 2016/0058641 A1
(43) Pub. Date: Mar. 3, 2016

(54) DEVICE FOR SUPPORTING A USER'S BODY

(71) Applicant: TheraTorr Medical, Inc., Gloucester, MA (US)

(72) Inventors: Timothy E. Moutafis, Beverly, MA (US); J. Christopher Flaherty, Auburndale, FL (US)

(73) Assignee: TheraTorr Medical, Inc., Gloucester, MA (US)

(21) Appl. No.: 14/776,202

(22) PCT Filed: Mar. 14, 2014
(86) PCT No.: PCT/US14/28835

§ 371 Date: Sep. 14, 2015

Related U.S. Application Data
(60) Provisional application No. 61/784,179, filed on Mar. 14, 2013.

(51) Int. Cl. A61G 7/057; A61B 5/11; A47C 27/08

(52) U.S. Cl. CPC A61G 7/05776; A61G 7/05769; A47C 27/085; A47C 27/088; A61B 5/1115; A47C 27/083; A47C 27/084

(57) ABSTRACT

A device for at least a portion of a patient's body is provided. The device comprises a plurality of bladders capable of containing a fluid and a plurality of support elements adjacent to and supporting the plurality of bladders. At least one support element is positioned adjacent to and supports each of the plurality of bladders. Bladders form a rolling diaphragm portion with the support elements such that when a force is applied to a bladder, the rolling diaphragm portion rolls along the associated support element.

### RELATED APPLICATIONS

The present application claims priority to U.S. Provisional Application Ser. No. 61/784,179, entitled “Device for Supporting a User's Body”, filed Mar. 14, 2013, which is incorporated by reference herein in its entirety.

The present application contains subject matter related to certain subject matter disclosed in U.S. Pat. No. 8,572,783 and U.S. patent application Ser. No. 14/012,606, filed Aug. 8, 2013 and currently pending. Each of the above indicated patent and patent application is incorporated by reference herein in its entirety.

### FIELD OF THE INVENTION

The present invention relates generally to devices for providing support to at least a portion of a user's body, and in particular to a device which may adjust the pressure on certain portions of the user's body.

### BACKGROUND OF THE INVENTION

Various types of support devices, such as mattresses, cushions and chairs, are known to support a user's body. A conventional mattress may include an array of spring elements to support a body. When a user lies on a conventional mattress, a number of the springs compress. As the level of compression increases, the resistive force in the springs increase as a result of user's weight on the mattress. This increased resistance tends to focus on protruding regions of patient anatomy which may cause lesions such as pressure ulcers, or other local circulatory problems, especially in bedridden patients. Protuberant regions of the anatomy are more prone to develop pressure sores because they tend to penetrate more deeply into mattresses, encountering greater forces than nearby regions and thus are more likely to have diminished local blood circulation.

Areas of a patient's body exposed to higher pressures when positioned on a support device, i.e., pressure points, are undesirable. Current methods to reduce pressure points on bedridden patients involve frequently moving or rotating the position of the patient on the support device so that a pressure point does not lead to the above-mentioned lesions. While this approach may be helpful, it requires someone, such as a nurse, to physically move the patient. This additional effort is time consuming, costly, and may also lead to injuring the nurse and/or the patient.

### SUMMARY

According to an aspect of the invention, a device for supporting at least a portion of a patient's body comprising a plurality of cells comprising a plurality of bladders capable of containing a fluid, the plurality of bladders including at least a first bladder and a second bladder; and a plurality of support elements adjacent to and supporting the plurality of bladders, such that at least one support element is positioned adjacent to and supports each of the plurality of bladders, where the plurality of support elements include at least a first support element and a second support element, with the first support element positioned adjacent to and supporting the first bladder and the second support element positioned adjacent to and supporting the second bladders. The first and second bladders each form a rolling diaphragm portion with the first and second support elements, respectively, such that when a force is applied to the first bladder, the rolling diaphragm portion of the first bladder rolls along the first support element decreasing the volume of the first bladder, and when a force is applied to the second bladder, the rolling diaphragm portion of the second bladder rolls along the second support element decreasing the volume of the second bladder.

The fluid can be selected from the group consisting of: a gas such as air; a liquid such as water; and combinations of these.

The internal pressure in one or more of the plurality of cells can be maintained at a pressure between 0.1 psig and 10 psig. The internal pressure in one or more of the plurality of cells can vary from cell to cell.

The plurality of cells can be arranged in a honeycomb configuration. The plurality of cells can be arranged in a rectangular configuration.

The plurality of cells can be arranged in a configuration providing a surface density ranging from 60% to 100%, for example 85%.

Each of the plurality of cells can comprise at least one of a round, square or hexagonal shape.

The plurality of cells can be grouped into one or more sections. For example, the plurality of cells can be grouped into four sections, where each section comprises 128 cells. The pressure can vary from section to section, for example, the pressure in a first section can be different from the pressure in a second, different section. The one or more sections can comprise one or more subsets. The one or more subsets can comprise at least one of one or more rows of cells, one or more columns of cells, or any geometric grouping of cells. The pressure can vary from one subset to a second, different subset.

The plurality of cells can be fluidically interconnected such that the pressure within a first cell is capable of reaching an equilibrium with the pressure within a second cell. The fluidic connections can be arranged in a geometric pattern.

The plurality of cells can comprise one or more sensors configured to provide feedback to a pressure distributor such that the pressure within a first cell reaches an equilibrium with the pressure within a second cell.

The space between adjacent cells of the plurality of cells ranges from 0.0 inches to 0.25 inches, for example approximately 0.1 inches.

A bladder diameter can range from 0.5 inches to 5.0 inches, for example 2.3 inches. A bladder wall thickness can range from 0.01 inches to 0.08 inches, for example, approximately 0.03 inches. A bladder functional length can range from 3.0 inches to 12.0 inches, for example approximately 5.0 inches. A bladder burst pressure can be greater than 80 mmHg, for example greater than 300 mmHg. A bladder operating strain at the rolling diaphragm portion can range from 5% to 100%, for example approximately 30%.

One or more of the plurality of bladders can comprise neoprene. One or more of the plurality of bladders can comprise a lubricious material. One or more of the plurality of bladders can comprise a material selected from the group consisting of: rubber; plastic; non-latex elastomer such as neoprene or urethane; polyethylene film; polypropylene blends; silicone; urethane laminates; latex laminates; and combinations of these.

One or more of the plurality of bladders can comprise a porous portion configured to controllably release a contained fluid.

One or more of the plurality of bladders can comprise a reinforcing layer and/or element.

One or more of the plurality of bladders can comprise a patient-contacting finish or layer. For example, the finish or layer can be selected from the group consisting of: foam; gel; padding; and combinations of these.

One or more of the plurality of bladders can comprise a coating, for example a lubricious coating. One or more of the plurality of bladders comprise a fabric coated with and/or molded to an elastomer. The fabric can be selected from the group consisting of: cotton; polyester; polyethylene; polypropylene; polyurethane; PTFE; FEP; nylon; a para-aramid synthetic fiber; and combinations of these. The elastomer can be selected from the group consisting of: a natural rubber; a synthetic compound such as a rubber having approximately a 30-90 shore D durometer; and combinations of these.

One or more of the plurality of bladders can comprise a conical shape. One or more of the plurality of bladders comprises a taper such that interference between a rolled portion and an unrolled portion is limited, for example a taper ranging from 0.2 degrees to 5.0 degrees such as a taper of 1.0 degree. One or more of the plurality of bladders can comprise a shape selected from the group consisting of: round; oval; square; rectangular; trapezoidal; polygonal; and combinations of these.

The device can comprise one or more sections where each section can comprise a plurality of bladders where a bladder material and/or size and/or shape varies from one section to a second, different section.

Each of the plurality of support elements can comprise a post configured to support an adaptor. The post diameter can range from 0.1 inches to 5.0 inches, for example approximately 0.57 inches. The post can comprise a lumen in fluid communication with its respective bladder. The lumen diameter can range from 0.03 inches to 4.9 inches, for example approximately 0.2 inches. The post can comprise aluminum. The post can comprise a material selected from the group consisting of: a plastic; a metal; ceramic; wood; and combinations of these. The post can be inflatable, and the post pressure can be greater than its respective bladder pressure. The post can comprise a top surface comprising a resilient material.

Each of the plurality of support elements can comprise an adaptor configured to initiate rolling of the rolling diaphragm portion of its respective bladder. The adaptor can comprise a tubular structure which circumferentially engages with a top portion of its respective post. The adaptor can comprise one or more notches such that the rolling diaphragm portion of its respective bladder rests in the one or more notches and wherein the bladder is secured to the adaptor via O-rings.

Each of the plurality of support elements can comprise a collar configured to provide a smooth transition as the rolling diaphragm portion of its respective bladder rolls from an adaptor to the collar. The collar can comprise a cylindrical tube which circumferentially engages a bottom portion of its respective adaptor and extends downward around its respective post. The collar can comprise a plastic material such as PVC pipe. The collar diameter can be approximately 90% of the adaptor diameter.

The device can further comprise a manifold configured to fluidly connect each cell of the plurality of cells to a pressure distributor such that the pressure in each cell can be independently controlled. Each cell of the plurality of cells can be independently connected to the manifold via its respective post. Each post can be electrically and/or mechanically connected to the manifold. Each post can be connected to the manifold via one more of a fiber optic connection; a fluid connection such as a hydraulic or pneumatic connection. The manifold can comprise the support elements. The device can further comprise a second manifold where a first grouping of the plurality of cells is connected to the first manifold and a second grouping of cells is connected to the second manifold. A grouping can comprise a section; a subset of a section; one or more rows; one or more columns; and/or a geometric grouping of cells.

The device can further comprise one or more valves configured to regulate the flow of fluid within the device. For example, the one or more valves can control the flow of fluid between each cell of the plurality of cells and a manifold. Additionally or alternatively, the one or more valves can control the flow of fluid between each cell of the plurality of cells and the surrounding environment. The one or more valves can be independently controlled. The one or more valves can be electronically controlled, and a controller can be configured to automatically or semi-automatically adjust the flow of fluid within the device. At least one of the one or more valves can comprise a pump configured to pump fluid to and/or from each cell of the plurality of cells, for example where the pump is configured to maintain a pressure within one or more cells of the plurality of cells at a different pressure than a manifold.

The device can further comprise a pressure distributor comprising a fluid delivery assembly configured to supply fluid to each cell of the plurality of cells. The pressure distributor can be configured to maintain an internal pressure of one or more of the plurality of cells between 0.1 psig to 10 psig. The pressure distributor can be configured to maintain an internal pressure of one or more of the plurality of cells at a pressure configured to prevent trauma to the surface of the patient's skin and/or to allow movement of the patient into or out of the device. The pressure distributor can be configured to independently control the internal pressure in each cell of the plurality of cells, for example where the internal pressure within each cell is less than 20 mmHg. Each cell of the plurality of cells can be fluidly connected to the pressure distributor via a respective fluid line.

The pressure distributor can be selected from the group consisting of: a gas compressor such as an air compressor; a fan; a liquid and/or gas pump; a fluid reservoir such as a pressurized liquid or gas reservoir; and combinations of these.

The pressure distributor can be further configured to supply fluid to each cell of the plurality of cells such that the device is maintained at a predetermined temperature.

The pressure distributor can comprise one or more functional elements selected from the group consisting of: a sensor; a transducer; a pressure regulator; a pump; a valve; and combinations of these. For example, the pressure regulator can be configured to maintain the pressure within one or more cells of the plurality of cells.

The pressure distributor can comprise one or more sensors selected from the group consisting of: a temperature sensor; a pressure sensor; an accelerometer; a gravitational sensor; an optical sensor; a magnetic sensor; and combinations of these.

The device can further comprise a controller configured to control one or more components of the device. For example, the controller can be configured to control a pressure distributor. The controller can be configured to independently control each cell of the plurality of cells. The device can comprise one or more sections, and the controller can be configured to independently control each of the one or more sections. At least one section of the one or more sections can comprise one or more subsets, and the controller can be configured to independently control each of the one or more subsets.

The controller can be automatic or manual. The controller can be configured to control a pressure within each cell of the plurality of cells at a constant or variable rate. The controller can communicate with the pressure distributor via a cable or wirelessly.

The controller can comprise a user interface comprising a graphical user interface and one or more controls. The controller can be configured to allow a user to enter one or more input parameters via one or more input components. The one or more input components can be selected from the group consisting of: touch screens, keyboards, joysticks, electronic mice, audio devices such as audio recorders; remote devices such as a hand-held wired or non-wired device, a phone, a mobile phone; and combinations of these. The one or more input parameters can be selected from the group consisting of: pressure and/or temperature to be maintained within each cell of the plurality of cells, section of cells, rows of cells, or any grouping of cells; duration of any pressure and/or temperature setting; information related to setting and/or controlling various modes; any patient parameter such as patient vital information; an alert such as an alert to notify a clinician of a particular patient activity or adverse condition such as a patient attempting to exit the device without required assistance, an increased site temperature which can be correlated to a potential pressure ulcer site, a pressure change other than a known or expected pressure change, or an instance where a bladder has made contact with a portion of its respective post or a manifold; operator specific information such as operator name and/or employee ID; facility specific information; environment specific information such as ambient pressure or humidity; security information such as a lock-out code; user permissions and/or restrictions such as permitted patient controls; and combinations of these. The device can further comprise one or more output components selected from the group consisting of: video displays; liquid crystal displays; alphanumeric displays; audio devices such as speakers; lights such as light emitting diodes; tactile alerts such as assemblies including a vibrating mechanism; and combinations of these.

The controller can be configured to generate one or more output signals configured to be received by one or more external electronic modules. The one or more output signals can be selected from the group consisting of: an electric current; electric signal; telephonic data stream; Bluetooth or other wireless signal; and combinations of these. The one or more external electronics modules can be selected from the group consisting of: an off-site alarm; computer processor; memory; video system; software; and combinations of these.

The controller can be configured to allow a user to initiate, modify and/or cease one or more device functions and/or modes. The user can be selected from the group consisting of a patient; clinician; physician; nurse; surgeon; any staff member of a hospital or health care facility; a family member; caregiver; and combinations of these.

A device function can comprise repositioning or rotating the patient.

The device can comprise a daytime/nighttime mode wherein the device comprises a first pressure during the daytime, and a different second pressure, during the nighttime.

The device can comprise a maximum flotation pressure mode wherein the pressure is controlled to simulate a sensation of weightlessness.

The device can comprise a passive restraint mode wherein the pressure is controlled so the patient is prevented from exiting a structure comprising the device.

The device can comprise an emergency mode wherein the pressure is controlled so the patient is elevated and the device is firm. In some embodiments, the device further comprises a second pressure regulator configured to control the emergency mode.

The device can comprise a stand-by mode wherein the pressure is controlled so that another mode can be entered quickly.

The device can comprise a timeout function configured to disable a mode and/or alert a user other than the patient if the mode is enabled for a duration exceeding a predetermined threshold.

The device can comprise a lockout function configured to prevent one or more users from initiating, modifying and/or stopping one or more device functions and/or modes.

The device can further comprise one or more sensors selected from the group consisting of: a pressure sensor; a temperature sensor; a vibration sensor; a strain sensor; an acoustic sensor; a magnetic sensor; an optical sensor; a moisture sensor; and combinations of these. The one or more sensors can be located at one or more of: any location on a bladder; at any location on a support element; on any location on a manifold.

The device can further comprise a position switch configured to indicate when a bladder of the plurality of bladders has traveled a predetermined distance. For example, the predetermined distance can be approximately one half of the total length of an inflated bladder, such as a predetermined distance of 2.5 inches of an inflated bladder comprising a total length of approximately 6 inches.

The device can further comprise a frame configured to support the device. The frame can comprise at least one side panel configured to maintain the position of each cell of the plurality of cells. The frame can comprise aluminum and/or plastic. The frame can comprise at least three sections, for example where the sections are adjustable and/or interchangeable. At least one section can comprise one or more subsections.

According to another aspect of the invention, a system for supporting at least a portion of a patient's body comprises a device comprising a plurality of cells comprising a plurality of bladders capable of containing a fluid, the plurality of bladders including at least a first bladder and a second bladder; and a plurality of support elements adjacent to and supporting the plurality of bladders, such that at least one support element is positioned adjacent to and supports each of the plurality of bladders, where the plurality of support elements include at least a first support element and a second support element, with the first support element positioned adjacent to and supporting the first bladder and the second support element positioned adjacent to and supporting the second bladder, and where the first and second bladders each forms a rolling diaphragm portion with the first and second support elements, respectively, such that when a force is applied to the first bladder, the rolling diaphragm portion of the first bladder rolls along the first support element decreasing the volume of the first bladder, and when a force is applied to the second bladder, the rolling diaphragm portion of the second bladder rolls along the second support element decreasing the volume of the second bladder. The system further comprises a pressure distributor configured to control a pressure in each of the plurality of cells; at least one manifold configured to fluidly connect each cell of the plurality of cells to the pressure distributor such that the pressure in each cell can be independently controlled; and a controller configured to control at least the pressure distributor.

The system can comprise the device as described above.

The system can further comprise a frame configured to support the device. The frame can comprise at least one side panel configured to maintain the position of each cell of the plurality of cells.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 is a schematic view of a system for supporting a patient's body, consistent with the present inventive concepts;

FIG. 2A is a cross sectional side view of a portion of a support device including a pair of cells each having a post comprising multiple components, consistent with the present inventive concepts;

FIG. 2B is a cross sectional side view of a portion of a support device including a pair of cells each having a post comprising a single component, consistent with the present inventive concepts;

FIGS. 3 and 3A are a perspective view and a top view, respectively, of a portion of a support device including an array of cells arranged in a honeycomb configuration, consistent with the present inventive concepts;

FIG. 4 is a perspective view of a bed frame comprising three sections;

FIG. 4A is a perspective view of one section of the bed frame of FIG. 4 comprising three sub-sections; and

FIG. 4B is a perspective view of a portion of a first support device comprising multiple cells and a second support device comprising a different material and/or construction than the first support device, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The present invention provides systems, devices and methods for supporting a portion of a patient's body, such as a patient being treated for a disease, disorder or injury. A system can include one or more support devices that are constructed and arranged to atraumatically support and/or reduce the pressure applied to support one or more particular areas of the patient's body, for example to treat and/or prevent the occurrence of pressure ulcers. It should be appreciated that in some embodiments, the support device can be part of and/or can form a mattress. In other embodiments, the support device can be part of and/or can form a chair, and in yet other embodiments, the support device can be part of and/or can form a cushion, pillow or other support device.

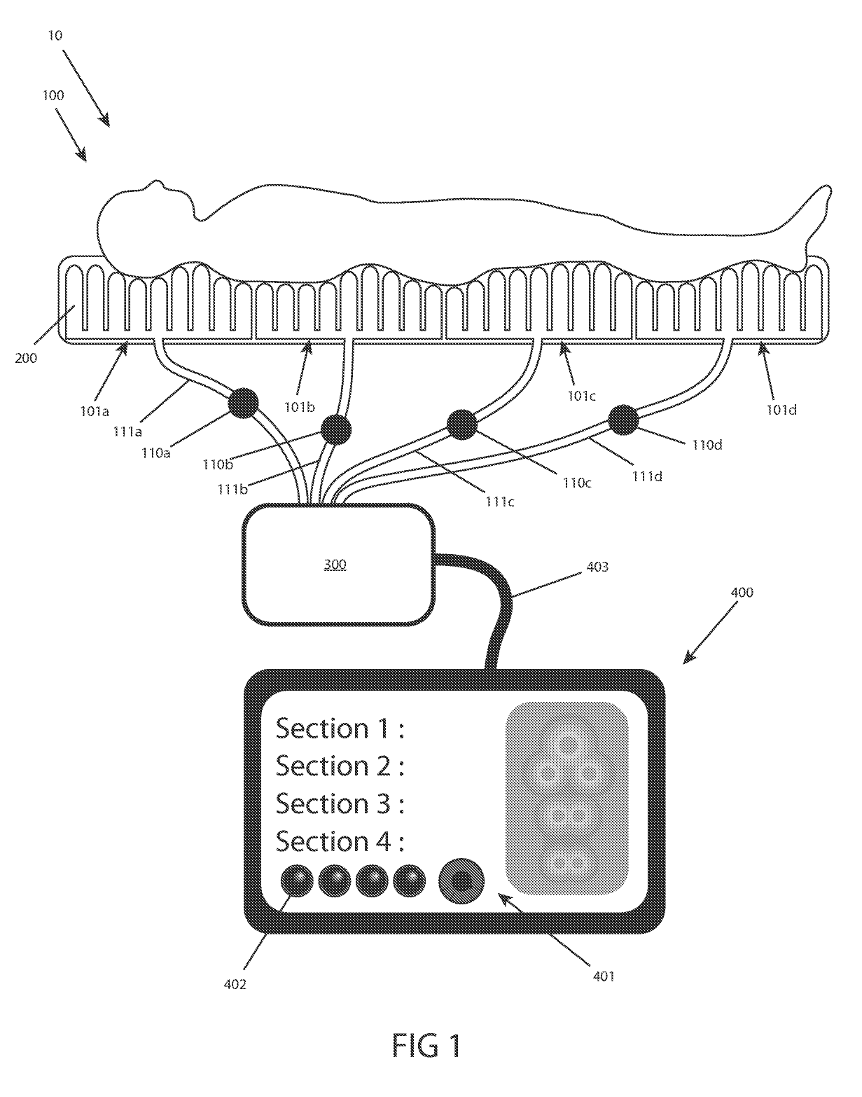

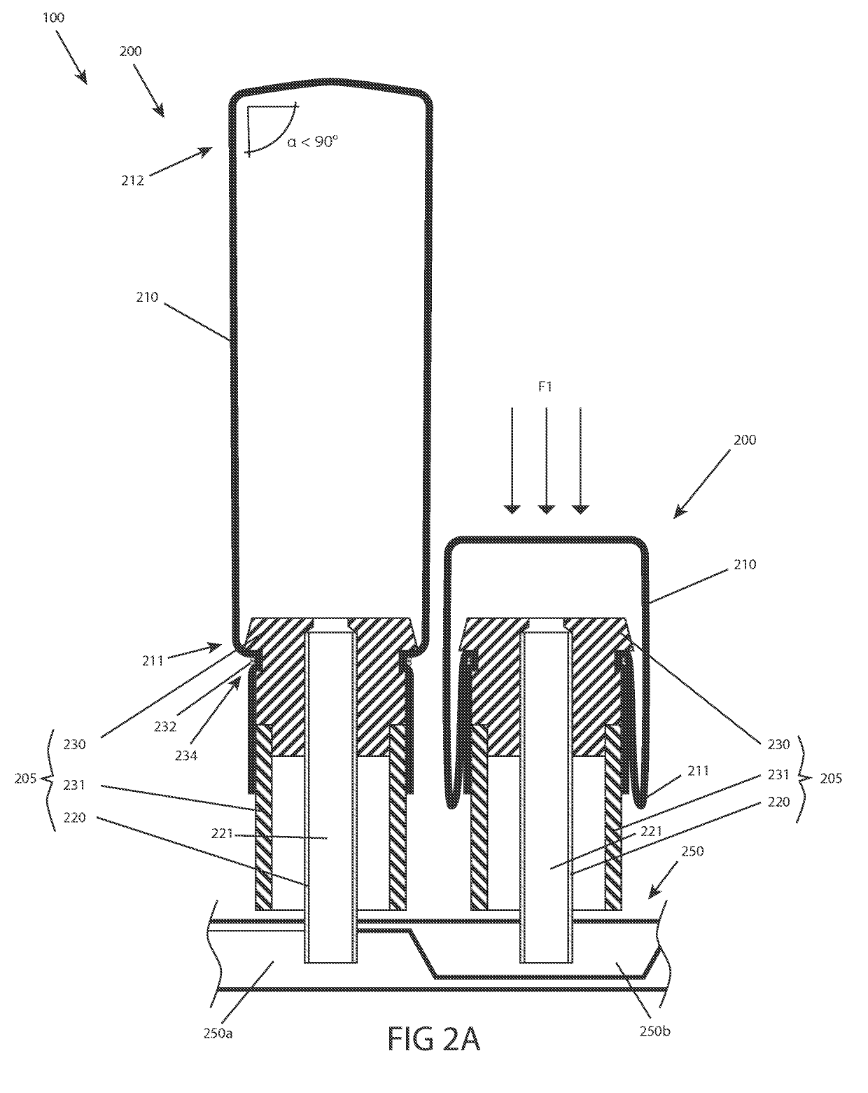

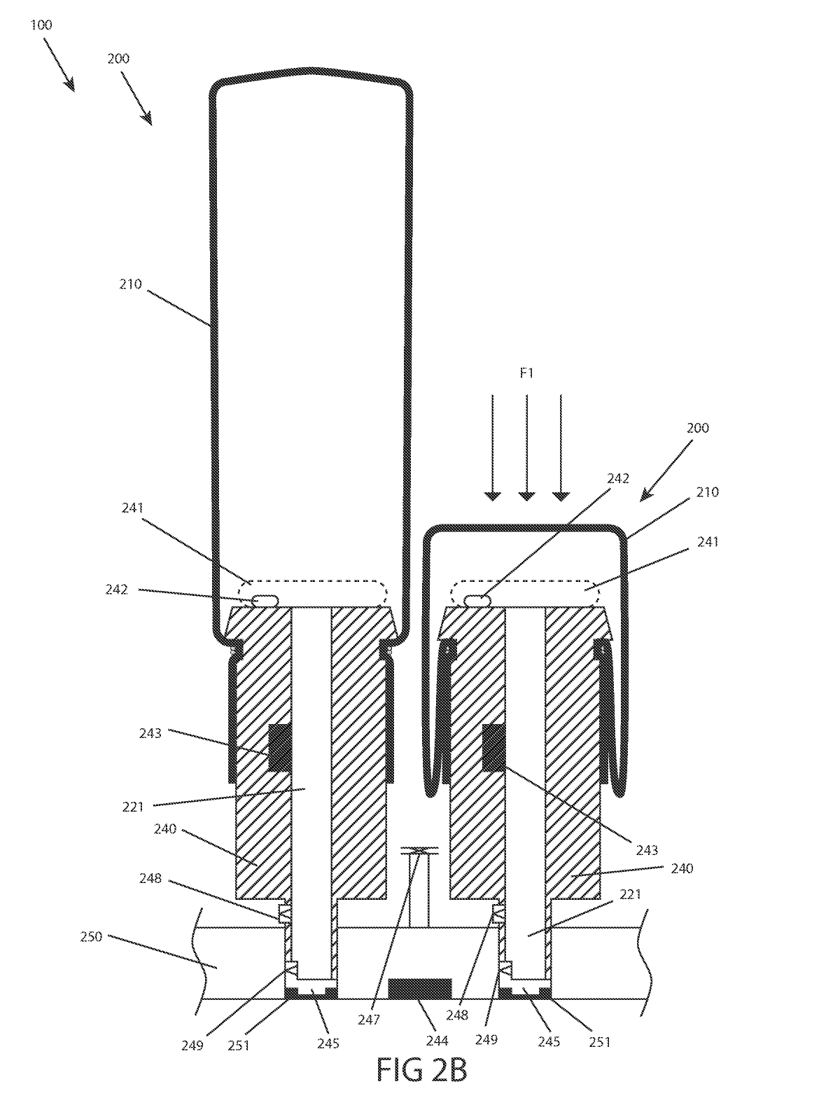

Referring now to FIG. 1, a schematic view of a system for supporting a patient's body is illustrated, consistent with the present inventive concepts. System 10 includes support device 100, pressure distributor 300 and controller 400. Support device 100 can include one or more sections, for example, as shown in the illustrated embodiment, support device 100 includes four sections 101a-d. Each section 101a-d can include any number of cells 200, each cell 200 comprising a bladder, including a rolling diaphragm portion, and a respective support element (bladder and support element described in detail in reference to FIGS. 2A and/or 2B herebelow). In response to a force applied to each cell 200, the volume of each cell 200 changes. In some embodiments, one or more cells 200 are constructed and arranged as described in Applicant's related U.S. Pat. No. 8,572,783, entitled “Device for Supporting A User's Body”, filed Feb. 22, 2010, the contents of which are incorporated by reference herein in its entirety.

Pressure distributor 300 comprises a fluid delivery assembly that can be configured to supply fluid to each section 101a-d via in-flow lines 111a-d, respectively, such that support device 100 can be maintained at a given pressure. In some embodiments, pressure distributor 300 is constructed and arranged to maintain one or more cells 200 at a low pressure, such as at a pressure less than 20 mmHggHG. The fluid provided by pressure distributor 300 can comprise air or other gases as well as liquids, such as water. In some embodiments, the provided fluid can be temperature controlled. Pressure distributor 300 can include a gas compressor such as an air compressor; a fan; a liquid and/or gas pump; a fluid reservoir such as a pressurized liquid or gas reservoir; and combinations of these. The pressure of fluid provided by pressure distributor 300 can be pressure-regulated, such as by one or more pressure-regulators included in pressure distributor 300 or another component of system 10. Pressure distributor 300 includes functional elements 110a-d which can comprise one or more functional elements selected from the group consisting of: a sensor; a transducer; a pressure regulator; a pump; a valve; and combinations of these. In some embodiments, functional elements 110a-d each comprise a pressure regulator fluidly connected to in-flow lines 111a-d, respectively, and configured to maintain the pressure within one or more cells 200 to a pre-determined level. Alternatively or additionally, one or more functional elements 110a-d comprise one or more sensors selected from the group consisting of: a temperature sensor; a pressure sensor; an accelerometer; a gravitational sensor; an optical sensor; a magnetic sensor; and combinations of these. Cells 200 can be fluidly connected to pressure distributor 300 via individual fluid lines (not shown), or via a manifold such as is shown in FIGS. 2A and 2B herebelow.

In some embodiments, the internal pressure within one or more cells 200 is maintained between approximately 0.1-1.0 psig (pressure above room atmospheric pressure). In another embodiment, the pressure within one or more cells 200 can be as much as approximately 1.0-10 psig, or more. In some embodiments, the internal pressure within each cell 200 can vary from cell to cell. In some embodiments, a larger pressure can be used to change dynamic properties of one or more portions of support device 100 (e.g. to make support device 100 firm). Larger pressures can also be delivered to cause support device 100 to expand, such as to elevate the patient (e.g. to elevate the top surface of cells 200 above a bed frame). For example, the pressure within one or more portions of support device 100 can be raised to stiffen support device 100 to ease the transfer of a patient onto or off of support device 100 (e.g. unassisted or with the help of a second person). In embodiments where support device 100 is made more firm to allow a patient onto support device 100, a subsequent reduction in pressure (e.g. to 0.1-1.0 psig) is performed to provided sustained, atraumatic support to the patient.

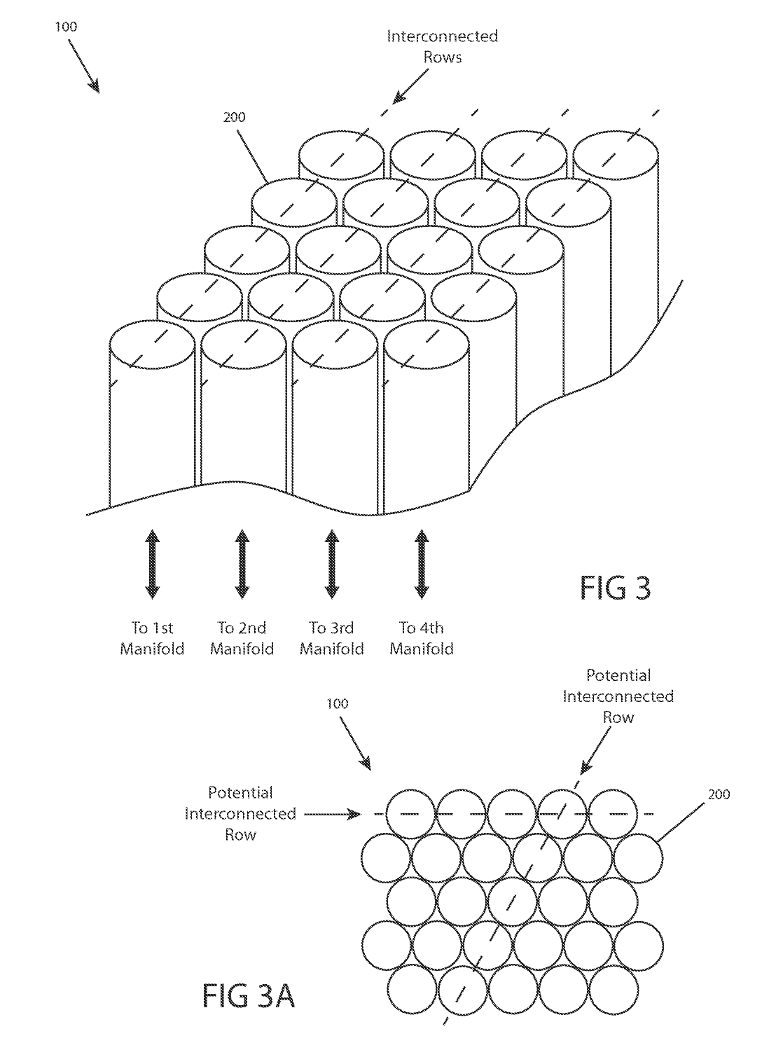

In some embodiments, the pressure is similar for each section 101a-d. In some embodiments, sections 101a-d can be independently set and/or controlled such that the pressure is similar or variable from section to section. For example, to ensure that a particular portion of a patient's body is provided a lower force than one or more other portions of the patient's body, one or more sections 101a,b,c and/or d can comprise a reduced pressure than other sections 101a, b, c, and/or d. In some embodiments, a first subset of cells 200 are at a first pressure and a second subset of cells 200 are at a second pressure, different than the first pressure. Three or more subsets of cells 200 can be included, such as at a third pressure different than the first or second pressures. A subset of cells 200 at a particular pressure (e.g. at the first or second pressures described above) can correlate to a particular subset of cells 200, such as sections 101a-d. A subset of cells 200 at a particular pressure can correlate to a row, column or other geometric grouping of cells 200 (such as is described in reference to FIGS. 3 and 3A herebelow). In some embodiments, the pressure between one or more subsets of cells 200 is varied with time, such as pressure that is alternated between at least a first pressure and a second pressure. For example the pressure of one or more sections 101a-d can be configured to change over timescales of seconds, minutes or hours, such as to improve local blood flow.

Cells 200 can be fluidically interconnected so that the pressure within a first cell 200 is capable of quickly reaching an equilibrium with the pressure within a second cell 200. For example, a fluid passage can extend through each cell 200, fluid passage not shown but described herebelow. In alternative embodiments, the pressure within a first pressure-regulated cell 200 reaches an equilibrium with the pressure within a pressure-regulated second cell 200 based on feedback received through one or more sensors, not shown but such as a pressure sensor integral to one or more cells 200.

Controller 400 is constructed and arranged to control pressure distributor 300 and/or one or more other assemblies or components of system 10. Controller 400 can include a user interface, comprising graphical user interface 401 and controls 402 which are configured to allow one or more users of system 10 to perform one or more functions such as the entering of one or more system input parameters. Additionally, controller 400 can be configured to allow a user to initiate, modify and cease any functions and/or modes of system 10. Users of system 10 can include a patient; clinician; physician; nurse; surgeon; any staff member of a hospital or health care facility; a family member; caregiver; and combinations of these. Controller 400 can include one or more user input components (e.g. touch screens, keyboards, joysticks, electronic mice, audio devices such as audio recorders and the like), and one or more user output components (e.g. video displays; liquid crystal displays; alphanumeric displays; audio devices such as speakers; lights such as light emitting diodes; tactile alerts such as assemblies including a vibrating mechanism; and the like). In some embodiments, controller 400 can include one or more output signals (e.g. an electric current; electric signal; telephonic data stream; Bluetooth or other wireless signal; and the like) which can be configured to be received by an external electronic module (i.e. an off-site alarm; computer processor; memory; video system; or the like). Output signals can be further configured to trigger an event selected from the group consisting of: activate an alarm; set an alert condition; trigger a recording mechanism; or combinations of these. Examples of system input parameters include but are not limited to: pressure and/or temperature to be maintained within individual cells 200, sections 101a-d, rows of cells 200, or any grouping of cells 200; duration of any pressure and/or temperature setting; information related to setting and/or controlling various modes of system 10; any patient parameter such as patient vital information; an alert such as an alert to notify a clinician of a particular patient activity or adverse system condition such as a patient attempting to exit a bed (e.g. a hospital bed including device 100) without required assistance, an increased site temperature which can be correlated to a potential pressure ulcer site, a pressure change other than a known or expected pressure change, or an instance where a bladder has made contact with a portion of its respective post or a manifold of system 10 (described further herebelow); operator specific information such as operator name and/or employee ID; facility specific information; environment specific information such as ambient pressure or humidity; security information such as a lock-out code; user permissions and/or restrictions such as permitted patient controls; and combinations of these. Controller 400 can be configured for manual and/or automatic control of device 100 or other portion of system 10. Additionally, controller 400 and/or one or more other components of system 10 can include an electronics module, such as an electronics module including a processor, memory, software, and the like.

Controller 400 can be in communication with pressure distributor 300 via cable 403 such that pressure distributor 300 regulates individual cells 200, subsets of cells 200 (e.g. a row or column of cells 200), and/or sections 101a-d. Regulation of individual cells 200 and/or sections 101a-d can include regulating pressure which can be constant or variable, for example with respect to time and/or with respect to the individual cells 200 and/or sections 101a-d. Alternatively, controller 400 can communicate with pressure distributor 300 via a wireless connection, for example via Bluetooth.

Controller 400 can be programmable such as to allow a user to store predetermined system settings for future use, for example system 10 can include various modes which can be set or otherwise controlled via user interface 401. In some embodiments, system 10 can include a daytime/nighttime mode in which a user can set device 100 at a first pressure during the daytime, and at a different second pressure, during the nighttime. Another mode can include a maximum flotation pressure mode where, for example pressure can be adjusted to be lower so as to simulate a sensation of “weightlessness”. Another mode can include a passive restraint mode, for example when a clinician may want to prevent a patient from exiting a hospital bed comprising support device 100. Another mode can include an emergency mode, for example, if a patient using device 100 requires CPR, pressure can be increased such that the patient is elevated and device 100 is extremely firm (e.g. to allow effective chest compressions). Another mode can include a stand-by mode, for example, device 100 pressure can be reduced such that device 100 is insufficient to support a patient, however device 100 pressure is “ready” to quickly enter another mode such as a patient operating mode or the emergency mode. Each mode can include a timeout function, for example if a particular mode has been enabled for a duration exceeding a predetermined threshold, the mode can be disabled and/or a clinician or hospital staff can be alerted. Alternatively or additionally, each mode can include a lockout function, for example if it is desirable to prevent a patient or a particular user from enabling or disabling a particular mode, user interface 401 can include password protected access to control the particular mode. Additionally or alternatively, pressure can be set and/or controlled to inflate and/or deflate certain cells 200 (e.g. rows, columns, etc of cells 200), for example to help reposition or rotate the patient. In some embodiments, each mode can be remotely controlled, for example, via a hand-held wired or non-wired device, a phone, a mobile phone, and the like.

Referring now to FIG. 2A, a cross sectional side view of a portion of a support device including a pair of cells each having a post comprising multiple components is illustrated, consistent with the present inventive concepts. Each cell 200 includes bladder 210 and a respective support element 205. In some embodiments, support element 205 comprises multiple attachable or pre-attached sections or components, such as post 220, adaptor 230, and collar 231 shown in FIG. 2A. Each bladder 210 is mechanically coupled to its respective post 220 via adaptor 230. Bladder 210 includes a circumferential segment, rolling diaphragm portion 211, configured to roll along a portion of the outer surface of adaptor 230 and the outer surface of collar 231. Rolling diaphragm portion 211 of bladder 210 can roll along adaptor 230 and collar 231 in response to a force applied to bladder 210, such as the force F1 shown in cell 200 to the right of the page. The position of bladder 210 with respect to adaptor 230 and collar 231 can affect the volume within the bladder 210. In one embodiment, the volume of bladder 210 decreases and increases as bladder 210 rolls along adaptor 230 and collar 231 in downward and upward directions of travel, respectively. Bladder 210 surrounds a fluid, where this fluid can enter and exit bladder 210 via a passage, such as lumen 221 of post 220 described herebelow. The fluid within bladder 210 can comprise air or other gases as well as liquids, such as water. The volume of bladder 210 is determined by the pressure at which the fluid is provided or maintained within bladder 210 and by the external forces applied to bladder 210 (e.g. to the top surface of bladder 210). The fluid within bladder 210 can be pressure controlled, such as via one or more pressure regulators described herein. In some embodiments, the fluid can also be temperature controlled.

Bladder 210 can comprise various materials, for example flexible and substantially fluid impermeable materials like rubber and/or various plastic materials. In some embodiments, bladder 210 comprises a non-latex elastomer, such as neoprene or urethane. In some embodiments, bladder 210 can include a thin walled material including a reinforcing layer and/or reinforcing elements. In some embodiments, bladder 210 comprises a fabric coated with and/or molded to an elastomer. For example, the fabric can be a cotton; polyester; polyethylene; polypropylene; polyurethane; PTFE; FEP; nylon or a para-aramid synthetic fiber such as KEVLAR® of DuPont, and the elastomer can be a natural rubber or a synthetic compound such as a rubber having approximately a 30-90 shore D durometer. In some embodiments, bladder 210 comprises one or more materials selected from the group consisting of: polyethylene film; polypropylene blends; silicone; urethane laminates; latex laminates; and combinations of these. Bladder 210 can comprise a lubricious material and/or a lubricious coating. Any or all surfaces of bladder 210 can include a patient-contacting finish or layer, which can include various types of foam, gel, and/or padding. Bladder 210 can comprise areas or portions that are porous, non-porous, or include one or more openings configured to controllably release the contained fluid.

The diameter of bladder 210 can range from approximately 0.5 inches to 5.0 inches, for example approximately 2.3 inches. The wall thickness of bladder 210 can range from approximately 0.01 inches to 0.08 inches, for example approximately 0.03 inches. Bladder wall thickness and material can be selected such that bladder 210 does not buckle, collapse, spontaneously inflate and blow up and/or prevent or otherwise hinder rolling at low pressures. The functional length of bladder 210 can range from approximately 3.0 inches to 12.0 inches, for example approximately 5.0 inches. The burst pressure of bladder 210 can be greater than approximately 80 mmHg, for example greater than approximately 300 mmHg. The operating strain of bladder 210 at rolling diaphragm portion 211 can range from approximately 5% to 100%, for example approximately 30%.

Bladder 210 can comprise a conical shape, with the cone expanding upwards, for example where bladder 210 comprises taper 212 configured to limit, e.g. reduce or eliminate, the interference between the rolled and unrolled portion of bladder 210. In some embodiments, the diameter of the upper portion of bladder 210 can be greater than the diameter of a lower portion of bladder 210 so as to create a taper ranging from approximately 0.2 degrees to 5.0 degrees, for example a taper of approximately 1.0 degree. Bladder 210 can comprise various cross sectional shapes including but not limited to: round; oval; square; rectangular; trapezoidal; polygonal; and combinations of these. The size, shape and material of bladders 210 can vary from section to section, for example sections 101a-d of FIG. 1 and/or can vary from cell 200 to cell 200, for example so as to vary the performance characteristics of device 100 or to reduce or increase the number of cells per unit area, for example to create individual areas, zones or containment of other zones of bladders.

Post 220 can include fluid flow lumen 221 in fluid communication with bladder 210 and manifold 250. Manifold 250 can comprise one or more sections, and in the illustrated embodiment, manifold includes two sections 250a and 250b. In these embodiments, device 100 can operate in a multiple pressure mode, for example, manifold 250a can comprise a first pressure, and manifold 250b can comprise a second, different pressure such that adjacent cells 200 comprise multiple pressures across device 100. Manifold 250a and 250b pressures can be set and/or controlled as described hereabove. In some embodiments, the pressure in manifolds 250a and/or 250b are alternated or switched over time, such that the corresponding cells 200 pressures vary between the first pressure and the second pressure.

Post 220 can comprise one or more materials selected from the group consisting of: a plastic; a metal; ceramic; wood; and combinations of these. In some embodiments, post 220 comprises aluminum. The diameter of post 220 can range from approximately 0.1 inches to 5.0 inches, for example approximately 0.57 inches. The diameter of lumen 221 can range from approximately 0.03 inches to 4.9 inches, for example approximately 0.2 inches.

In one embodiment, posts 220 are inflatable, and can be held at a pressure greater than the pressure within bladders 210. As noted above, the pressure in bladders 210 can be maintained at a pressure less than 1 psig. In some embodiments, if contact between the patient's body occurs, posts 220 can be cushioned. For example, a resilient material can be included on the top surfaces of bladders 210, within bladders 210 and/or as part of posts 220 and/or the top surfaces of adaptors 230 to provide this cushioning effect.

Adaptor 230 comprises a tubular structure which circumferentially engages the top portion of post 220. Collar 231 comprises a cylindrical tube which circumferentially engages the bottom portion of adaptor 230 and extends downward around post 220. Adaptor 230 is constructed and arranged to initiate rolling of bladder 210 onto a portion of adaptor 230 and collar 231. Collar 231 comprises a diameter such that bladder 210 is provided a smooth transition as it rolls from adaptor 230 to collar 231. Bladder 210 is attached to adaptor 230 via one or more O-rings 232 (two shown) that surround bladder 210 at rolling diaphragm portion 211 and rest in notches 234 of adaptor 230. Bladder 210 can roll between approximately 0.0 inches (from the O-ring circumference) and approximately half of the total length of the inflated bladder, for example approximately 2.5 inches for an inflated bladder comprising a length of approximately 6 inches (bladder 210 length can include an additional 0.5 inches to 1 inch in length to reduce the tension placed upon bladder 210 when bladder 210 has traveled its maximum distance, i.e. at full compression). In some embodiments, system 10 includes a sensor configured to indicate bladder 210 position with respect to manifold 250, such as is described in reference to FIG. 2B herebelow.

In another embodiment manifold 250 comprises one or more sheets, for example vacuum formed sheets. In this embodiment, posts 220 can be formed and molded directly onto a sheet eliminating the need for a post 220, adaptor 230, and collar 231. Alternatively, posts 220 can be glued or welded to a sheet, for example via heat or radiofrequency (RF) welding. In some embodiments, posts 220 are removable, for example for replacement and/or to change the size of bladder 210.

In some embodiments, the diameter of adaptor 230 is between approximately 10% and 30% less than the mandrel rolled large diameter, for example approximately 10% less than the mandrel rolled large diameter. In some embodiments, the diameter of collar 231 is between approximately 30% and 100% of the diameter of adaptor 230, for example approximately 90% of the diameter of adaptor 230. Collar 231 can comprise a plastic material, for example PVC pipe.

In some embodiments, support element 205 is integrated into manifold 250. In some embodiments, support element 205 is a separate component of device 100.

Referring now to FIG. 2B, a cross sectional side view of a portion of a support device including a pair of cells each having a support element comprising a single component is illustrated, consistent with the present inventive concepts. Cells 200 comprise similar components and functionality as cells 200 of FIG. 2A, except that the support element 205 including post 220, adaptor 230 and collar 231 of FIG. 2A now comprises a single component, support element 240. Alternatively or additionally, cells 200 of FIG. 2B can comprise one or more additional functional elements as are described herebelow.

Each support element 240 can independently attach to manifold 250 such as via one or more mechanical (e.g. fluid) and/or electrical connections. Support element 240 is electrically attached to manifold 250 via receptor 251 and plug 245. By individually connecting each support element 240, each cell 200 can be individually controlled, for example the pressure within each cell 200 can be individually controlled via a controller, such as controller 400 of FIG. 1. Additionally or alternatively, data collected from any of the below described sensors 242, 243, 244 and switch 247 can be displayed on a user interface via the electrical connections, and/or can be used in one or more control algorithms such as to adjust pressure in a closed-loop fashion. Other connections can include connections such as a fiber optic connection; a fluid connection such as a hydraulic or pneumatic connection; and combinations of these.

Device 100 can include one or more valves, such as valves 248 and 249 configured to control the flow of fluid within device 100, for example to control pressure within device 100. Valve 248 is positioned such that the flow of fluid can be controlled between cell 200 and its surrounding environment (e.g. the room in which device 100 is placed). Valve 249 is positioned such that the flow of fluid can be controlled between cell 200 and manifold 250. Valves 248 and 249 can be independently controllable with respect to one another. Valves 248 and/or 249 can comprise electronically controllable valves, such as to be adjusted automatically or semi-automatically such as via a controller such as controller 400 of FIG. 1. Valve 249 or another component of cell 200 can comprise a pump, such as a pump constructed and arranged to pump fluid to and/or from cell 200, such as to independently adjust the pressure maintained within cell 200. In some instances, it may be desirable to maintain the pressure within bladder 210 at a pressure lower than the pressure of manifold 250, or otherwise change the pressure to a level different than manifold 250 pressure. In these instances, fluid can be added to (e.g. pumped) or removed from bladder 210 via a pumping mechanism as described hereabove.

Alternatively, fluid can be removed from bladder 210 via a controllable pressure regulator and valve and/or via a pump (e.g. into the room environment and/or back into manifold 250). In some embodiments, fluid is released from bladder 210 into the surrounding room environment via valve 248, such as when a rapid lowering of bladder 210 pressure is desired and/or when manifold 250 pressure is above a desired bladder 210 pressure. In some embodiments, system 10 can include a second, separate system comprising a controllable pressure regulator and valve and/or pump that can be configured, for example to transition device 100 into the emergency mode.

Device 100 can include one or more sensors, such as sensors 242, 243 and 244. Sensor 242 is positioned at the top of support element 240 as shown, such as to measure a support element 240 parameter such as contact of bladder 210 with support element 240. Sensor 243 is positioned adjacent to lumen 221, such as to measure a lumen 221 parameter such as the pressure within lumen 221. Sensor 244 is positioned in manifold 250, such as to measure a manifold 250 parameter such as manifold 250 pressure. Sensor 242, 243 and/or 244 can comprise one or more sensors selected from the group consisting of: pressure sensors; temperature sensors; vibration sensors; strain sensors; acoustic sensors; magnetic sensors; optical sensors; moisture sensors; and combinations of these. In some embodiments, sensor 242 can include a pressure sensor or a magnetic sensor configured to indicate bladder position with respect to support element 240. In some embodiments, sensor 243 can include a pressure sensor configured to indicate pressure within lumen 221, such as a pressure that correlates to pressure within bladder 210. Alternatively or additionally, sensor 243 can include a temperature sensor configured to indicate temperature within bladder 210, for example so as to monitor the temperature of a fluid in bladder 210. Similarly, sensor 244 can include a pressure sensor and/or temperature sensor configured to indicate the pressure and/or temperature of a fluid within manifold 250.

As describe above, bladder 210 rolls along support element 240. In the illustrated embodiment of FIG. 2B, device 100 includes a position switch 247 positioned in the excursion pathway of bladder 210. Switch 247 is constructed and arranged to indicate when bladder 210 has traveled a desired maximum distance, so as to prevent contact between bladder 210 and a top surface of support element 240. The maximum distance can be approximately half of the total length of an inflated bladder, for example 2.5 inches for an inflated bladder comprising a total length of approximately 6 inches (bladder 210 length can include an additional 0.5 inches to 1 inch in length to reduce the tension placed upon bladder 210 when bladder 210 has traveled its maximum distance, i.e. at full compression). Given the described cell properties, cell 200 pressure remains constant as bladder 210 compresses.

In some embodiments, support element 240 can include padding 241 on its top surface. Padding can comprise foam; gel; or other suitable material and can be configured to provide an atraumatic top surface to support element 240. Padding 241 can be included on any or all support elements 240 of device 100.

Referring now to FIGS. 3 and 3A, a perspective view and a top view, respectively, of a portion of a support device including an array of cells is illustrated, consistent with the present inventive concepts. Device 100 includes multiple cells 200, such as cells 200 described in reference to FIGS. 2A and/or 2B hereabove. Cells 200 can be fluidly connected in one or more patterns, such as fluidly connected rows, columns or other geometric patterns. Spacing between adjacent cells 200 can range from 0.0 inches to 0.25 inches, for example 0.10 inches. In the embodiment of FIG. 3A, cells 200 comprise a round cross sectional shape, having a diameter of approximately 2 inches and are arranged in a honeycomb pattern which can provide a surface density, depending on the spacing ranging from 60% to 100%, for example 85%. “Surface density” as used herein, is the ratio of surface area occupied by cells 200 divided by the total surface area. In some embodiments, cells 200 can be arranged in a rectangular matrix and can comprise a square cross sectional shape, and the spacing between adjacent cells 200 can be reduced such that the surface density of device 100 can be up to 100%. Cells 200 can comprise any shape and or spacing to achieve the desired surface density of device 100. In some embodiments, cells 200 comprise a hexagonal shape and are arranged in a honeycomb pattern. Adjacent bladders may contact one another or not, and the amount of contact can be adjusted to achieve a desired contact area and/or contact pressure of device 100. In some embodiments, bladders 210 may be separated by a material such as foam, gel, cotton batting and/or padding.

Pressure and/or temperature of cells 200 can be regulated by a pressure distributor, for example pressure distributor 300 of FIG. 1. In some embodiments, the pressure distributor can be configured to supply fluid to device 100 such that support device 100 can be maintained at a given pressure, for example such that device 100 comprises a low interface pressure.

In some embodiments, the pressure is maintained and/or modified as described in reference to FIG. 1 hereabove, such as to prevent trauma to the surface of the patient's skin and/or to allow movement of the patient into or out of support device 100. Pressure can be maintained and/or modified for the entire set of cells 200, for one or more subsets of cells 200 and/or for one or more individual cells 200, as has been described hereabove.

In some embodiments, cells 200 can be interconnected, for example in rows, such that rows of cells can be independently controlled. In the embodiments of FIGS. 3A, cells 200 are arranged in a honeycomb fashion, creating horizontal rows (from left to the right of the page as shown) and diagonal rows (from the top to the bottom of the page as shown). In one example, a first diagonal row can be connected to a first manifold comprising a first pressure, and a second diagonal row can be connected to a second, different manifold comprising a second, different pressure (manifolds not shown, but described in FIGS. 1, 2A and 2B hereabove). Continuing with this example, a third diagonal row can be connected to a third manifold comprising a third pressure similar to the first pressure, and a fourth diagonal row can be connected to a fourth, different manifold comprising a fourth pressure similar to the second pressure. Alternatively, cells 200 can be arranged in a row and column configuration or any suitable configuration based on the desired pressure of a particular discrete area or section of device 100. Different pressures in different sets of cells 200 can be alternated between the sets of cells 200, such as to allow and/or cause increased blood flow in the patient, and/or to provide other therapeutic benefit.

It should be appreciated that any combination of cells, groups of cells, rows of cells, either horizontal or diagonal, can comprise same or different pressures and/or temperatures, either constant or variable with time.

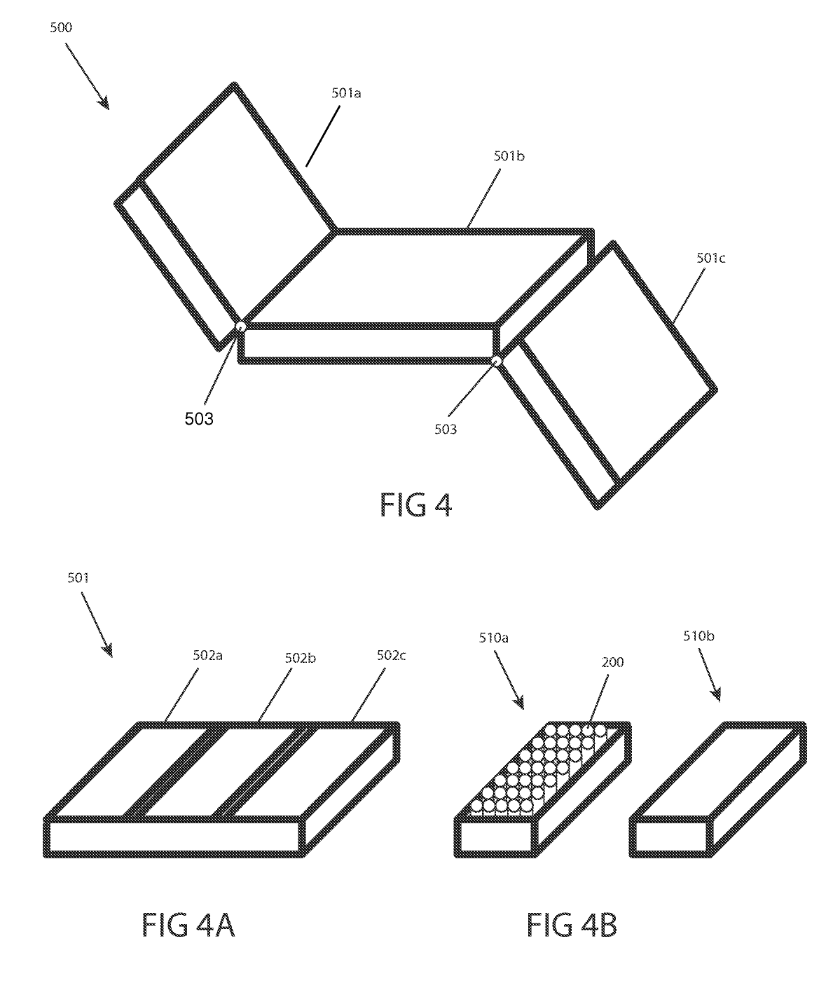

Referring now to FIG. 4, a perspective view of a bed frame comprising three sections is illustrated, consistent with the present inventive concepts. Frame 500 comprises upper section 501a, middle section 501b and lower section 501c, and each section can be rotatably adjusted with respect to one another as shown, for example via at least one hinge 503. Alternatively, frame 500 is not adjustable. Although three sections are illustrated, frame 500 can comprise any number of sections. Frame 500, regardless of number of sections included, comprises at least one side panel configured to stabilize and maintain the position of each cell 200 (as shown in FIG. 4B). As an example, frame 500 comprises section 501a, where section 501a comprises four side panels where the side panels stabilize and maintain the position of cells 200 with respect to frame 500 and with respect to all other cells within frame 500. Similarly, sections 501b and 501c comprise side panels. Any number of sections or subsections (shown in 4A) comprising side panels can be used. Additionally, frame can comprise panels positioned at any location within frame configured to further stabilize and maintain the position of cells 200, for example a panel positioned diagonally within frame 500. Frame 500 can comprise any rigid material, for example aluminum and/or plastic. Frame 500 can comprise a cushioning layer on its outer surface. For example, the layer can be selected from the group consisting of: foam; gel; padding; and combinations of these.

Referring additionally to FIG. 4A, a perspective view of one section of the bed frame of FIG. 4 comprising three sub-sections is illustrated, consistent with the present inventive concepts. Section 501 can comprise two or more sub-sections, such as the three sub-sections 502a-c shown. Section 501 of FIG. 4A can be used as subsection 501a, 501b and/or 501c of FIG. 4 described hereabove.

Referring additionally to FIG. 4B, a perspective view of a portion of a first support device comprising multiple cells and a second support device comprising a different material and/or construction, consistent with the present inventive concepts. First support device 510a comprises multiple cells 200, such as multiple cells 200 arranged in a honeycomb pattern with alternating rows attached to two independent pressure manifolds as described hereabove.

Second support device 510b comprises a different configuration, such as a standard spring mattress, a foam pad, a gel pad; a different pattern of cells 200, or other support device. In some embodiments, second support device 510b has a lower cost than first support device 510a, and combinations of support devices 510a and 510b are used to reduce overall cost to provide a complete bed or other body support device. A total of three of first support device 510a and second support device 510b can be used to create subsection 501a of FIG. 4A, such as two first support devices 510a and one support device 510b, in any pattern. Two or more of any combination of support devices 510a and 510b can be used to create any of subsections 501a, 501b, and 501c of FIG. 4, in any pattern. Combinations of support devices 510a and/or 510b can be used to reduce cost, meet specific patient conditions, or otherwise. By way of example only, if a patient has traumatized skin at a certain location on the body, first support device 510a comprising multiple cells 200 can be positioned at that body location while support device 510b positioned in other body locations.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

We claim:

1. A device for supporting at least a portion of a patient's body, the device comprising:
a plurality of cells comprising:
a plurality of bladders capable of containing a fluid, the plurality of bladders including at least a first bladder and a second bladder; and
a plurality of support elements adjacent to and supporting the plurality of bladders, such that at least one support element is positioned adjacent to and supports each of the plurality of bladders,
wherein the plurality of support elements include at least a first support element and a second support element, with the first support element positioned adjacent to and supporting the first bladder and the second support element positioned adjacent to and supporting the second bladder, and wherein the first and second bladders each forms a rolling diaphragm portion with the first and second support elements, respectively, such that when a force is applied to the first bladder, the rolling diaphragm portion of the first bladder rolls along the first support element decreasing the volume of the first bladder, and when a force is applied to the second bladder, the rolling diaphragm portion of the second bladder rolls along the second support element decreasing the volume of the second bladder.

2. The device of any other device claim wherein the fluid is selected from the group consisting of: a gas such as air; a liquid such as water; and combinations thereof.

3. The device of any other device claim wherein the internal pressure in one or more of the plurality of cells is maintained at a pressure between 0.1 psig and 10 psig.

4. The device of any other device claim wherein the internal pressure in one or more of the plurality of cells varies from cell to cell.

5. The device of any other device claim wherein the plurality of cells are arranged in a honeycomb configuration.

6. The device of any other device claim wherein the plurality of cells are arranged in a rectangular configuration.

7. The device of any other device claim wherein the plurality of cells are arranged in a configuration providing a surface density ranging from 60% to 100%.

8. The device of claim 7 wherein the plurality of cells are arranged in a configuration providing a surface density approximating 85%.

9. The device of any other device claim wherein each of the plurality of cells comprise at least one of a round, square or hexagonal shape.

10. The device of any other device claim wherein the plurality of cells are grouped into one or more sections.

11. The device of claim 10 wherein the plurality of cells are grouped into four sections.

12. The device of claim 11 wherein each section comprises 128 cells.

13. The device of claim 10 wherein pressure varies from one section to a second, different section.

14. The device of claim 10 wherein the one or more sections comprise one or more subsets.

15. The device of claim 14 wherein the one or more subsets comprise at least one of one or more rows of cells, one or more columns of cells, or any geometric grouping of cells.

16. The device of claim 15 wherein pressure varies from one subset to a second, different subset.

17. The device of any other device claim wherein the plurality of cells are fluidically interconnected such that the pressure within a first cell is capable of reaching an equilibrium with the pressure within a second cell.

18. The device of claim 17 wherein the fluidic connections are arranged in a geometric pattern.

19. The device of any other device claim wherein the plurality of cells comprise one or more sensors configured to provide feedback to a pressure distributor such that the pressure within a first cell reaches an equilibrium with the pressure within a second cell.

20. The device of any other device claim wherein a space between adjacent cells of the plurality of cells ranges from 0.0 inches to 0.25 inches.

21. The device of claim 20 wherein the space between adjacent cells of the plurality of cells approximates 0.10 inches.

22. The device of any other device claim wherein a bladder diameter ranges from 0.5 inches to 5.0 inches.

23. The device of claim 22 wherein a bladder diameter approximates 2.3 inches.

24. The device of any other device claim wherein a bladder wall thickness ranges from 0.01 inches to 0.08 inches.

25. The device of claim 24 wherein a bladder wall thickness approximates 0.03 inches.

26. The device of any other device claim wherein a bladder functional length ranges from 3.0 inches to 12.0 inches.

27. The device of claim 26 wherein a bladder functional length approximates 5.0 inches.

28. The device of any other device claim wherein a bladder burst pressure is greater than 80 mmHg.

29. The device of claim 28 wherein a bladder burst pressure is greater than 300 mmHg.

30. The device of any other device claim wherein a bladder operating strain at the rolling diaphragm portion ranges from 5% to 100%.

31. The device of claim 30 wherein a bladder operating strain at the rolling diaphragm portion approximates 30%.

32. The device of any other device claim wherein one or more of the plurality of bladders comprise neoprene.

33. The device of any other device claim wherein one or more of the plurality of bladders comprise a lubricious material.

34. The device of any other device claim wherein one or more of the plurality of bladders comprise a material selected from the group consisting of: rubber;
plastic; non-latex elastomer such as neoprene or urethane; polyethylene film;
polypropylene blends; silicone; urethane laminates; latex laminates; and
combinations thereof.

35. The device of any other device claim wherein one or more of the plurality of bladders comprise a porous portion configured to controllably release a contained fluid.

36. The device of any other device claim wherein one or more of the plurality of bladders comprise a reinforcing layer and/or element.

37. The device of any other device claim wherein one or more of the plurality of bladders comprise a patient-contacting finish or layer.

38. The device of claim 37 wherein the patient-contacting finish or layer comprises a finish or layer selected from the group consisting of: foam; gel; padding; and combinations thereof.

39. The device of any other device claim wherein one or more of the plurality of bladders comprise a coating.

40. The device of claim 39 wherein the coating comprises a lubricious coating.

41. The device of claim 39 wherein one or more of the plurality of bladders comprise a fabric coated with and/or molded to an elastomer.

42. The device of claim 41 wherein the fabric is selected from the group consisting of: cotton; polyester; polyethylene; polypropylene; polyurethane; PTFE; FEP;
nylon; a para-aramid synthetic fiber; and combinations thereof.

43. The device of claim 42 wherein the elastomer is selected from the group consisting of: a natural rubber; a synthetic compound such as a rubber having approximately a 30-90 shore D durometer; and combinations thereof.

44. The device of any other device claim wherein one or more of the plurality of bladders comprises a conical shape.

45. The device of any other device claim wherein one or more of the plurality of bladders comprises a taper such that interference between a rolled portion and an unrolled portion is limited.

46. The device of claim 45 wherein the taper ranges from 0.2 degrees to 5.0 degrees.

47. The device of claim 46 wherein the taper approximates 1.0 degree.

48. The device of any other device claim wherein one or more of the plurality of bladders comprises a shape selected from the group consisting of: round; oval; square; rectangular; trapezoidal; polygonal; and combinations thereof.

49. The device of any other device claim wherein the device comprises one or more sections wherein each section comprises a plurality of bladders and wherein a bladder material and/or size and/or shape varies from one section to a second, different section.

50. The device of any other device claim wherein each of the plurality of support elements comprises a post configured to support an adaptor.

51. The device of claim 50 wherein the post diameter ranges from 0.1 inches to 5.0 inches.

52. The device of claim 51 wherein the post diameter approximates 0.57 inches.

53. The device of claim 50 wherein the post comprises a lumen in fluid communication with its respective bladder.

54. The device of claim 53 wherein the lumen diameter ranges from 0.03 inches to 4.9 inches.

55. The device of claim 54 wherein the lumen diameter approximates 0.2 inches.

56. The device of claim 50 wherein the post comprises aluminum.

57. The device of claim 50 wherein the post comprises a material selected from the group consisting of: a plastic; a metal; ceramic; wood; and combinations thereof.

58. The device of claim 50 wherein the post is inflatable and wherein the post pressure is greater than its respective bladder pressure.

59. The device of claim 50 wherein the post comprises a top surface comprising a resilient material.

60. The device of any other device claim wherein each of the plurality of support elements comprises an adaptor configured to initiate rolling of the rolling diaphragm portion of its respective bladder.

61. The device of claim 60 wherein the adaptor comprises a tubular structure which circumferentially engages with a top portion of its respective post.

62. The device of claim 61 wherein the adaptor comprises one or more notches such that the rolling diaphragm portion of its respective bladder rests in the one or more notches and wherein the bladder is secured to the adaptor via Orings.

63. The device of any other device claim wherein each of the plurality of support elements comprises a collar configured to provide a smooth transition as the rolling diaphragm portion of its respective bladder rolls from an adaptor to the collar.

64. The device of claim 63 wherein the collar comprises a cylindrical tube which circumferentially engages a bottom portion of its respective adaptor and extends downward around its respective post.

65. The device of claim 63 wherein the collar comprises a plastic material such as PVC pipe.

66. The device of claim 63 wherein the collar diameter is approximately 90% of the adaptor diameter.

67. The device of any other device claim further comprising a manifold configured to fluidly connect each cell of the plurality of cells to a pressure distributor such that the pressure in each cell can be independently controlled.

68. The device of claim 67 wherein each cell of the plurality of cells is independently connected to the manifold via its respective post.

69. The device of claim 68 wherein each post is electrically and/or mechanically connected to the manifold.

70. The device of claim 68 wherein each post is connected to the manifold via one more of a fiber optic connection; a fluid connection such as a hydraulic or pneumatic connection.

71. The device of claim 67 wherein the manifold comprises the plurality of support elements.

72. The device of claim 67 further comprising a second manifold where a first grouping of the plurality of cells is connected to the first manifold and a second grouping of cells is connected to the second manifold.

73. The device of claim 72 wherein a grouping comprises a section; a subset of a section; one or more rows; one or more columns; and/or a geometric grouping of cells.

74. The device of any other device claim further comprising one or more valves configured to control the flow of fluid within the device.

75. The device of claim 74 wherein the one or more valves control the flow of fluid between one or more cells of the plurality of cells and the surrounding environment.

76. The device of claim 74 wherein the one or more valves control the flow of fluid between each cell of the plurality of cells and a manifold.

77. The device of claim 74 wherein the one or more valves are independently controlled.

78. The device of claim 74 wherein the one or more valves are electronically controlled and wherein a controller is configured to automatically or semi-automatically adjust the flow of fluid within the device.

79. The device of claim 74 wherein at least one of the one or more valves comprises a pump configured to pump fluid to and/or from each cell of the plurality of cells.

80. The device of claim 79 wherein the pump is configured to maintain a pressure within one or more cells of the plurality of cells at a different pressure than a manifold.

81. The device of any other device claim further comprising a pressure distributor comprising a fluid delivery assembly configured to supply fluid to each cell of the plurality of cells.

82. The device of claim 81 wherein the pressure distributor is configured to maintain an internal pressure of one or more of the plurality of cells between 0.1 psig to 10 psig.

83. The device of claim 81 wherein the pressure distributor is configured to maintain an internal pressure of one or more of the plurality of cells at a pressure configured to prevent trauma to the surface of the patient's skin and/or to allow movement of the patient into or out of the device.

84. The device of claim 81 wherein the pressure distributor is configured to independently control the internal pressure in each cell of the plurality of cells.

85. The device of claim 84 wherein the internal pressure in each cell of the plurality of cells is less than 20 mmHg.

86. The device of claim 84 wherein the internal pressure in one or more of the plurality of cells is less than 20 mmHg.

87. The device of claim 81 wherein the pressure distributor is selected from the group consisting of: a gas compressor such as an air compressor; a fan; a liquid and/or gas pump; a fluid reservoir such as a pressurized liquid or gas reservoir; and combinations thereof.

88. The device of claim 81 wherein the pressure distributor is further configured to supply fluid to each cell of the plurality of cells such that the device is maintained at a predetermined temperature.

89. The device of claim 81 wherein the pressure distributor comprises one or more functional elements selected from the group consisting of: a sensor; a transducer; a pressure regulator; a pump; a valve; and combinations thereof.

90. The device of claim 89 wherein the pressure regulator is configured to maintain the pressure within one or more cells of the plurality of cells.

91. The device of claim 81 wherein the pressure distributor comprises one or more sensors selected from the group consisting of: a temperature sensor; a pressure sensor; an accelerometer; a gravitational sensor; an optical sensor; a magnetic sensor; and combinations thereof.

92. The device of claim 81 wherein each cell of the plurality of cells is fluidly connected to the pressure distributor via a respective fluid line.

93. The device of any other device claim further comprising a controller configured to control one or more components of the device.

94. The device of claim 93 wherein the controller is configured to control a pressure distributor.

95. The device of claim 94 wherein the controller is configured to independently control each cell of the plurality of cells.

96. The device of claim 94 wherein the device comprises one or more sections, and wherein the controller is configured to independently control each of the one or more sections.

97. The device of claim 96 wherein at least one section of the one or more sections comprises one or more subsets, and wherein the controller is configured to independently control each of the one or more subsets.

98. The device of claim 94 wherein the controller is automatic or manual.

99. The device of claim 94 wherein the controller is configured to control a pressure within each cell of the plurality of cells at a constant or variable rate.

100. The device of claim 94 wherein the controller communicates with the pressure distributor via a cable or wirelessly.

101. The device of claim 93 wherein the controller comprises a user interface comprising a graphical user interface and one or more controls.

102. The device of claim 101 wherein the controller is configured to allow a user to enter one or more input parameters via one or more input components.

103. The device of claim 102 wherein the one or more input components are selected from the group consisting of: touch screens, keyboards, joysticks, electronic mice, audio devices such as audio recorders; remote devices such as a hand-held wired or non-wired device, a phone, a mobile phone; and combinations thereof.

104. The device of claim 102 wherein the one or more input parameters are selected from the group consisting of: pressure and/or temperature to be maintained within each cell of the plurality of cells, section of cells, rows of cells, or any grouping of cells; duration of any pressure and/or temperature setting; information related to setting and/or controlling various modes; any patient parameter such as patient vital information; an alert such as an alert to notify a clinician of a particular patient activity or adverse condition such as a patient attempting to exit the device without required assistance, an increased site temperature which can be correlated to a potential pressure ulcer site, a pressure change other than a known or expected pressure change, or an instance where a bladder has made contact with a portion of its respective post or a manifold; operator specific information such as operator name and/or employee ID; facility specific information; environment specific information such as ambient pressure or humidity; security information such as a lock-out code; user permissions and/or restrictions such as permitted patient controls; and combinations thereof.

105. The device of claim 102 further comprising one or more output components selected from the group consisting of: video displays; liquid crystal displays; alphanumeric displays; audio devices such as speakers; lights such as light emitting diodes; tactile alerts such as assemblies including a vibrating mechanism; and combinations thereof.

106. The device of claim 101 wherein the controller is configured to generate one or more output signals configured to be received by one or more external electronic modules.

107. The device of claim 106 wherein the one or more output signals is selected from the group consisting of: an electric current; electric signal; telephonic data stream; Bluetooth or other wireless signal; and combinations thereof.

108. The device of claim 106 wherein the one or more external electronics modules are selected from the group consisting of: an off-site alarm; computer processor; memory; video system; software; and combinations thereof.

109. The device of claim 93 wherein the controller is configured to allow a user to initiate, modify and/or cease one or more device functions and/or modes.

110. The device of claim 109 wherein a device function comprises repositioning or rotating the patient.

111. The device of claim 109 wherein the user is selected from the group consisting of a patient; clinician; physician; nurse; surgeon; any staff member of a hospital or health care facility; a family member; caregiver; and combinations thereof.

112. The device of claim 109 wherein the device comprises a daytime/nighttime mode wherein the device comprises a first pressure during the daytime, and a different second pressure, during the nighttime.

113. The device of claim 109 wherein the device comprises a maximum flotation pressure mode wherein the pressure is controlled to simulate a sensation of weightlessness.

114. The device of claim 109 wherein the device comprises a passive restraint mode wherein the pressure is controlled so the patient is prevented from exiting a structure comprising the device.

115. The device of claim 109 wherein the device comprises an emergency mode wherein the pressure is controlled so the patient is elevated and the device is firm.

116. The device of claim 115 further comprising a second pressure regulator configured to control the emergency mode.

117. The device of claim 109 wherein the device comprises a stand-by mode wherein the pressure is controlled so that another mode can be entered quickly.

118. The device of claim 109 wherein the device comprises a timeout function configured to disable a mode and/or alert a user other than the patient if the mode is enabled for a duration exceeding a predetermined threshold.

119. The device of claim 109 wherein the device comprises a lockout function configured to prevent one or more users from initiating, modifying and/or stopping one or more device functions and/or modes.

120. The device of any other device claim further comprising one or more sensors selected from the group consisting of: a pressure sensor; a temperature sensor; a vibration sensor; a strain sensor; an acoustic sensor; a magnetic sensor; an optical sensor; a moisture sensor; and combinations thereof.

121. The device of claim 120 wherein the one or more sensors are located at one or more of: any location on a bladder; at any location on a support element; on any location on a manifold.

122. The device of any other device claim further comprising a position switch configured to indicate when a bladder of the plurality of bladders has traveled a predetermined distance.

123. The device of claim 122 wherein the predetermined distance is approximately one half of the total length of an inflated bladder.

124. The device of claim 123 wherein the predetermined distance is 2.5 inches of an inflated bladder comprising a total length of approximately 6 inches.

125. The device of any other device claim further comprising a frame configured to support the plurality of cells and the plurality of support elements.

126. The device of claim 125 wherein the frame comprises at least one side panel configured to stabilize and/or maintain the position of each cell of the plurality of cells.

127. The device of claim 126 wherein the frame comprises aluminum and/or plastic.

128. The device of claim 126 wherein the frame comprises a layer on its outer surface selected from the group consisting of: foam; gel; padding; and combinations thereof.

129. The device of claim 125 wherein the frame comprises at least three sections.

130. The device of claim 129 wherein the sections are adjustable.

131. The device of claim 129 wherein the sections are interchangeable.

132. The device of claim 129 wherein at least one section comprises one or more subsections.

133. A system for supporting at least a portion of a patient's body, the system comprising:
a device comprising:
a plurality of cells comprising:
a plurality of bladders capable of containing a fluid, the plurality of bladders including at least a first bladder and a second bladder; and
a plurality of support elements adjacent to and supporting the plurality of bladders, such that at least one support element is positioned adjacent to and supports each of the plurality of bladders,
wherein the plurality of support elements include at least a first support element and a second support element, with the first support element positioned adjacent to and supporting the first bladder and the second support element positioned adjacent to and supporting the second bladder,
wherein the first and second bladders each forms a rolling diaphragm portion with the first and second support elements, respectively, such that when a force is applied to the first bladder, the rolling diaphragm portion of the first bladder rolls along the first support element decreasing the volume of the first bladder, and when a force is applied to the second bladder, the rolling diaphragm portion of the second bladder rolls along the second support element decreasing the volume of the second bladder;
a pressure distributor configured to control a pressure in each of the plurality of cells;
at least one manifold configured to fluidly connect each cell of the plurality of cells to the pressure distributor such that the pressure in each cell can be independently controlled; and
a controller configured to control at least the pressure distributor.

134. The system of any other system claim wherein the system comprises a device of any of claims 1 through 131.

135. The system of any other system claim wherein the system further comprises a frame configured to support the device.

136. The system of claim 135 wherein the frame comprises at least one side panel configured to stabilize and/or maintain the position of each cell of the plurality of cells.

137. A device as shown in reference to the figures.

138. A system as shown in reference to the figures.
